(19) United States
(12) Patent Application Publication
(10) Pub. No.: US 2012/0190806 A1
(43) Pub. Date: Jul. 26, 2012

(54) Phosphine Borane Compounds Comprising Imidazol Groups And Method For Producing Phosphine Borane Compounds Comprising Imidazol Groups

(75) Inventors: Christoph Jäkel, Limburgerhof (DE); Peter Hofmann, Heidelberg (DE); Corina Scriban, Cambridge (GB); Patrick Hanno-Igels, Heidelberg (DE)

(73) Assignee: BASF SE, Ludwigshafen (DE)

(21) Appl. No.: 13/388,096

(22) PCT Filed: Jul. 30, 2010
(86) PCT No.: PCT/EP2010/061075

§ 371 Date: Apr. 16, 2012

(30) Foreign Application Priority Data
Jul. 31, 2009 (EP) 09166945.7

(51) Int. Cl. C08F 4/80; C07C 231/14; C07F 15/04; C07F 9/6506; C07F 15/00

(52) U.S. Cl. 526/131; 548/110; 548/112; 548/103; 560/155

(57) ABSTRACT

The present invention relates to imidazolo-containing phosphinoborane compounds, to optically active ligands prepared using them, to transition metal complexes which comprise such ligands, and to catalysts which comprise such transition metal complexes. The present invention further relates to the particular processes for preparing the phosphinoborane compounds, the optically active ligands, the transition metal complexes and the catalysts, and to the use of the catalysts for organic transformation reactions. The present invention further relates to a process for preparing optically active ligands comprising imidazolo-containing phosphorus compounds using imidazolo-containing phosphinoborane compounds.

The present invention relates to imidazolo-containing phosphinoborane compounds, to optically active ligands prepared using them, to transition metal complexes which comprise such ligands, and to catalysts which comprise such transition metal complexes. The present invention further relates to the particular processes for preparing the phosphinoborane compounds, the optically active ligands, the transition metal complexes and the catalysts, and to the use of the catalysts for organic transformation reactions. The present invention further relates to a process for preparing optically active ligands comprising imidazolo-containing phosphorus compounds using imidazolo-containing phosphinoborane compounds.

Organic transformation reactions, for example asymmetric hydrogenations, hydroformylations or polymerizations, are important reactions in the large-scale chemical industry. These organic transformation reactions are predominantly catalyzed homogeneously.

In order to obtain catalysts with high activity and enantioselectivity, it is frequently necessary to perform complicated, multistage and hence costly syntheses. Usually, the syntheses of the ligands by which the reactivity of the metal complexes can be tailored are the most difficult step. The search for ligands which are based on readily available starting material and/or are preparable by a simple synthesis method is consequently a permanent task in reducing costs in chemical catalysis.

In order, for example, to be able to control the stereoselectivity in an asymmetric hydrogenation or the tacticity in a polymerization, chiral ligands are required. One starting skeleton for chiral ligands might be NHCP ligands, which have potential by virtue of a sterically protected phosphorus atom and a carbene as a strong σ-donor.

In spite of this starting basis, the prior art has to date disclosed only achiral and a few chiral NHCP ligands. Organometallics 2007, volume 26, pages 253 to 263, Chemistry-A European Journal 2007, volume 13, pages 3652 to 3659, Journal of Organometallic Chemistry 2005, volume 690, pages 5948 to 5958 and Organometallics 2003, volume 22, pages 4750 to 4758 describe, for example, achiral NHCP ligands of the following type:

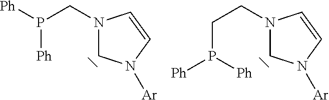

where “Ph” represents phenyl and “Ar” represents 2,6-diisopropylphenyl or 2,4,6-trimethylphenyl.

Applications of these systems in catalytic transformations are described in Organic Letters 2001, volume 3, pages 1511 to 1514, Advanced Synthesis & Catalysis 2004, volume 346, pages 595 to 598, Inorganica Chimica Acta 2004, volume 357, pages 4313 to 4321, Journal of Organometallic Chemistry 2006, volume 691, pages 433 to 443, and Organometallics 2005, volume 24, pages 4241 to 4250.

Only a few chiral NHCP ligands have been described to date; for instance, the prior art ((a) S. Nanchen, A. Pfaltz//Helvetica Chimica Acta 89 (2006) 1559-1573, (b) E. Bappert, G. Helmchen//Synlett 10 (2004)1789-1793, (c) H. Lang, J. Vittal, P-H. Leung//Journal of the Chemical Society, Dalton Transactions (1998) 2109-2110) discloses ligands which feature an ethano bridge between the phosphorus atom and the nitrogen atom of the imidazole group element. Further NHCP ligands have been described in the following literature: ((a) H Seo, H. Park, B. Y. Kim, J. H. Lee, S. U. Son, Y. K. Chung//Organometallics (22) 2003, 618-620, (b) T. Focken, G. Raabe, C. Bolm//Tetrahedron: Asymmetry 15 (2004) 1693-1706, (c) T. Focken, J. Rudolph, C. Bolm//Synthesis (2005) 429-436, (d) R. Hodgson, R. E. Douthwaite//Journal of Organometallic Chemistry 690 (2005) 5822-5831). In this case too, a relatively long bridge has been selected between the phosphorus atom and a nitrogen atom of the imidazole group element. However, all chiral NHCP ligands described to date exhibit only moderate enantioselectivities.

Achiral NHCP ligands typically have two identical substituents on the phosphorus atom. Chiral NHCP ligands, in contrast, may possess different ligands on the phosphorus atom. If this is the case, the phosphorus atom itself becomes a chiral center. These chiral compounds are particularly advantageously suitable as ligands for catalysts in enantioselective catalysis, for example enantioselective hydrogenations. For enantioselective catalysis, it is possible to use essentially only those chiral compounds which are present in substantially enantiomerically pure form, i.e. only in the form of one enantiomer.

Unfortunately, the compounds prepared by exchange of the symmetric substituents on the phosphorus atom, which now have a chiral phosphorus atom, cannot be utilized for enantioselective catalysis, since these compounds formed after conventional synthesis would have both enantiomeric forms of ligands in equal amounts, as the racemate.

In addition, the prior art (WO 03/022812 A1; WO 2006/087333 A1; WO 03/037835 A2; EP 1 182 196 A1) includes ionic liquids having the following imidazolium cations of the formula below, which, however, do not comprise NHCP combinations:

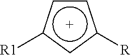

where the R1 radical is selected from the group consisting of a) hydrogen, b) linear or branched, saturated or unsaturated, aliphatic or alicyclic alkyl groups having 1 to 20 carbon atoms, c) heteroaryl groups, heteroaryl-C₁-C₆-alkyl groups having 3 to 8 carbon atoms in the heteroaryl radical and at least one heteroatom which is selected from N, O and S and may be substituted by at least one group selected from C₁-C₆-alkyl groups and/or halogen atoms, d) aryl groups, aryl-C₁-C₆-alkyl groups which have 5 to 16 carbon atoms in the aryl radical and may optionally be substituted by at least one C₁-C₆-alkyl group and/or a halogen atom, and the R radical is selected from the group consisting of a) linear or branched, saturated or unsaturated, aliphatic or alicyclic alkyl groups having 1 to 20 carbon atoms, b) heteroaryl-C₁-C₆-alkyl groups having 3 to 8 carbon atoms in the heteroaryl radical and at least one heteroatom which is selected from N, O and S and may be substituted by at least one group selected from C₁-C₆-alkyl groups and/or halogen atoms, c) aryl-C₁-C₆-alkyl groups which have 5 to 16 carbon atoms in the aryl radical and may optionally be substituted by at least one C₁-C₆-alkyl group and/or a halogen atom.

The documents cited disclose various preparation methods for the ionic liquids described. It is also stated that the ionic liquids are used as solvents, as phase transfer catalysts, as extractants, as heat carriers, as operating fluid in process or working machines, or as an extraction medium or as a constituent of the reaction medium for extractions of polarizable impurities/substrates.

International patent application PCT/EP2009/051677 describes imidazolo-containing phosphorus compounds, ligands prepared therefrom, transition metal complexes and catalysts with the following formula:

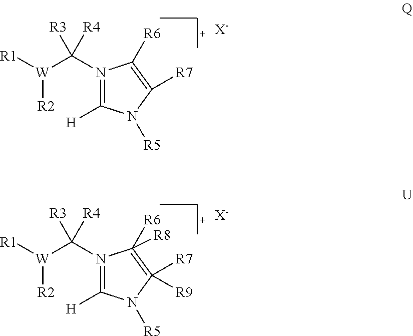

in which W is phosphorus or phosphite.

It is accordingly an object of the present invention to provide novel optically active ligands and catalysts based thereon. These ligands should be synthesizable with industrially inexpensively available starting materials and reagents and without considerable apparatus complexity. The ligands or the catalyst should preferably be preparable in a one-stage process. In particular, both enantiomers of the particular ligands should be preparable with similar efficiency. Moreover, the ligands or the catalysts prepared therefrom should be suitable for use in organic transformation reactions with high stereoselectivity and/or good regioselectivity. Furthermore, the organic transformation reactions should have a yield comparable to the prior art.

It has been found that, surprisingly, the catalysts prepared from the NHCP ligands characterized in detail below have a good efficiency compared to the prior art with significantly lower synthesis costs. The NHCP ligands are not only simple and inexpensive to prepare, but are also exceptionally robust. Moreover, it is even possible to prepare both enantiomers with a low level of complexity.

The process according to the invention allows the synthesis of chiral compounds with different amounts of the two enantiomers. More particularly, the process according to the invention allows differentiation of these enantiomers to such a degree that the formation of the undesired enantiomer is suppressed so substantially that it has only a negligible influence in the synthetic application.

For the purpose of illustrating the present invention, the expression “alkyl” comprises straight-chain and branched alkyl groups. They are preferably straight-chain or branched C₁-C₂₀-alkyl, more preferably C₁-C₁₂-alkyl, especially preferably C₁-C₈-alkyl and very especially preferably C₁-C₄-alkyl groups. Examples of alkyl groups are especially methyl, ethyl, propyl, isopropyl, n-butyl, 2-butyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, n-heptyl, 2-heptyl, 3-heptyl, 2-ethylpentyl, 1-propylbutyl, n-octyl, 2-ethylhexyl, 2-propylheptyl, nonyl, decyl.

The expression “alkyl” also comprises substituted alkyl groups which have generally 1, 2, 3, 4 or 5, and preferably 1, 2 or 3 substituents and more preferably 1 substituent. These are preferably selected from alkoxy, cycloalkyl, aryl, hetaryl, hydroxyl, halogen, NE¹E², NE¹E²E³⁺, carboxylate and sulfonate. A preferred perfluoroalkyl group is trifluoromethyl.

In the context of the present invention, the expression “aryl” comprises unsubstituted and also substituted aryl groups, and is preferably phenyl, tolyl, xylyl, mesityl, naphthyl, fluorenyl, anthracenyl, phenanthrenyl or naphthacenyl, more preferably phenyl or naphthyl, where these aryl groups, in the case of substitution, may generally bear 1, 2, 3, 4 or 5 and preferably 1, 2 or 3 substituents and more preferably 1 substituent, selected from the groups of alkyl, alkoxy, carboxylate, trifluoromethyl, sulfonate, NE¹E², alkylene-NE¹E², nitro, cyano and halogen. A preferred perfluoroaryl group is pentafluorophenyl.

In the context of this invention, carboxylate and sulfonate preferably represent a derivative of a carboxylic acid function and of a sulfonic acid function respectively, especially a metal carboxylate or sulfonate, a carboxylic ester or sulfonic ester function or a carboxamide or sulfonamide function. These include, for example, the esters with C₁-C₄-alkanols such as methanol, ethanol, n-propanol, isopropanol, n-butanol, sec-butanol and tert-butanol.

The above illustrations of the terms “alkyl” and “aryl” apply correspondingly to the terms “alkoxy” and “aryloxy”.

In the context of the present invention, the term “acyl” represents alkanoyl or aroyl groups having generally 2 to 11 and preferably 2 to 8 carbon atoms, for example the formyl, acetyl, propanoyl, butanoyl, pentanoyl, hexanoyl, heptanoyl, 2-ethylhexanoyl, 2-propylheptanoyl, benzoyl or naphthoyl group.

The E¹ to E³ radicals are each independently selected from hydrogen, alkyl, cycloalkyl and aryl. The NE¹E² group is preferably N,N-dimethylamino, N,N-diethylamino, N,N-dipropylamino, N,N-diisopropylamino, N,N-di-n-butylamino, N,N-di-t-butylamino, N,N-dicyclohexylamino or N,N-diphenylamino.

Halogen represents fluorine, chlorine, bromine and iodine, preferably fluorine, chlorine and bromine.

In the context of the invention, the term “leaving group” represents those structural elements which can be substituted by attack of or reaction with nucleophiles. These leaving groups are generally known to those skilled in the art and, for example, chlorine, bromine, iodine, trifluoroacetyl, acetyl, benzoyl, tosyl, nosyl, triflate, nonaflate, camphor-10-sulfonate and the like are used.

In the description part of the present patent application, for simplification, both enantiomers are also included when only one enantiomer is specified.

The invention provides imidazolo-containing phosphinoborane compounds of the general formula I or II:

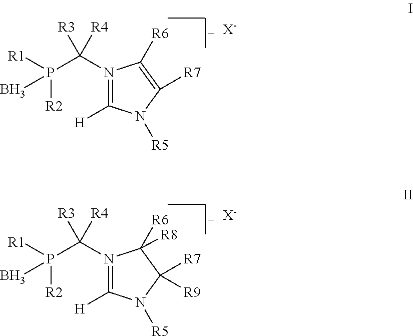

in which
R1 and R2 are different radicals and are selected from the group consisting of alkyl and alkyl (variant α)
or
R1 and R2 are different radicals and are selected from the group consisting of alkyl and aryl (variant β),
R3 and R4 are each identical or different radicals selected from the group consisting of hydrogen, alkyl and aryl,
R5 is alkyl or aryl,
R6 and R7 are each identical or different radicals selected from the group consisting of hydrogen, alkyl, aryl and a 6-membered aliphatic or aromatic ring,
R8 and R9 are each independently hydrogen or alkyl,
X is a leaving group.

In the case in which the R1 and R2 radicals are a combination of alkyl and alkyl (variant α), one alkyl radical is preferably adamantyl, tert-butyl, sec-butyl or isopropyl, especially tert-butyl, and the other alkyl radical is methyl, ethyl, propyl, butyl, pentyl or hexyl, especially methyl or ethyl, more preferably methyl.

In the case in which the R1 and R2 radicals are a combination of aryl and alkyl (variant β), the aryl radical is preferably phenyl, tolyl, xylyl, mesityl, naphthyl, fluorenyl, anthracenyl, especially phenyl, and the alkyl radical is methyl, adamantyl, tert-butyl, sec-butyl, isopropyl, especially tert-butyl and methyl.

The combination of alkyl and alkyl (variant α) is preferred over the combination of alkyl and aryl (variant β).
R3 and R4 are preferably each independently hydrogen, methyl, ethyl or benzyl, especially hydrogen.
R5 is preferably methyl, ethyl, isopropyl, tert-butyl, adamantyl, mesityl, 2,6-diisopropylphenyl, phenyl, tolyl, xylyl, naphthyl, fluorenyl, anthracenyl, especially methyl, isopropyl, tert-butyl, adamantyl, mesityl and 2,6-diisopropylphenyl.
R6 and R7 are preferably each independently hydrogen or a 6-membered aromatic ring.
R8 and R9 are preferably each independently hydrogen, alkyl, especially methyl, ethyl, isopropyl, tert-butyl, adamantyl, a (CH₂)₄ chain or aryl, especially phenyl, tolyl, xylyl, mesityl, naphthyl, fluorenyl, anthracenyl. More preferably, R8 and R9 are each independently hydrogen, phenyl or a (CH₂)₄ chain.

The inventive imidazolo-containing phosphinoborane compounds are advantageously optically active.

Particular preference is given either to both enantiomers or diastereomers, which have originated from the enantiomeric form of the cation, and their enantiomers of the following imidazolo-containing phosphinoborane compounds:

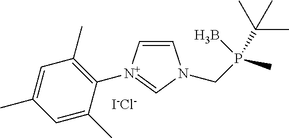

1-{[tert-butyl(methyl)phosphinoboranyl]methyl}-3-tert-butyl-1H-imidazol-3-ium chloride/iodide

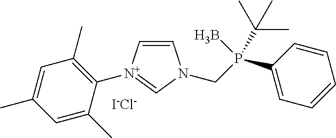

1-{[tert-butyl(phenyl)phosphinoboranyl]methyl}-3-(2,4,6-trimethylphenyl)-1H-imidazol-3-ium chloride/iodide

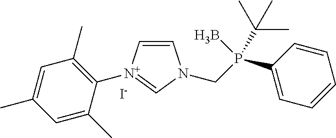

3-mesityl-1-((tert-butyl(phenyl)phosphinoboranyl)methyl)-1H-imidazol-3-ium iodide

The present invention further relates to a process for preparing imidazolo-containing phosphinoborane compounds of the general formula I or II, which comprises reacting compounds of the general formula A

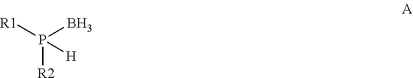

with compounds of the general formula B or C

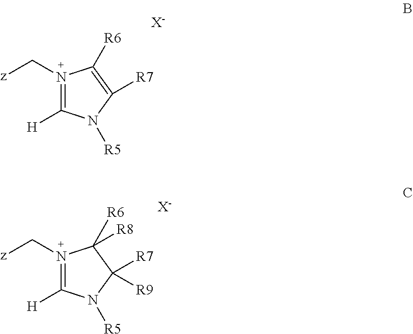

at a temperature of −80° C. to 40° C. in the presence of at least one lithium ion-containing base, for example alkyllithium compounds, using one or more solvents.

The present invention further relates to a process for preparing optically active imidazolo-containing phosphinoborane compounds of the general formula I or II, which comprises reacting compounds of the general formula A

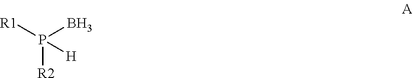

with compounds of the general formula B or C

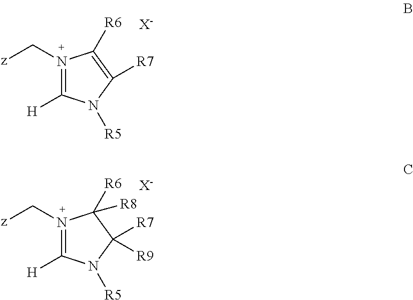

at a temperature of −80° C. to 40° C. in the presence of at least one lithium ion-containing base and of at least one chiral auxiliary, for example (-)-sparteine or a corresponding surrogate, such as (1R,2S,9S)-11-alkyl-7,11-diazatricyclo[7.3.1.0^{2,7}]tridecane, (1R,2S,9S)-11-methyl-7,11-diazatricyclo[7.3.1.0^{2,7}]tridecane, where alkyl in this case is preferably methyl, ethyl, butyl, benzyl, isopropyl, 2,2,2-trimethylethyl, 1-cyclopropylmethyl or 1-cyclohexylmethyl, using one or more solvents.

Using compound B results in a reaction to give compound I, and using compound C results in a reaction to give compound II.

Preference is given to performing the reaction at a temperature of −40 to 40° C., especially at a temperature of −20 to 30° C. The reaction time is typically 1 to 48 hours, preferably 4 to 24 hours. The optimal reaction time can be determined by the person skilled in the art by simple routine tests. The solvents used may be all solvents known to those skilled in the art; particularly suitable solvents are THF, diethyl ether or tert-butyl methyl ether.

If the compounds are to be prepared in enantiomerically enriched form, it is advantageous even before the reaction of compound B or C with compound A to react the compound A with at least one chiral (optically active) auxiliary, in the presence of a lithium ion-containing base. Thereafter, the reaction with B or C can be performed as described above.

Preference is given to performing this pretreatment as described below: compound A is reacted with at least one optically active auxiliary and at least one lithium ion-containing base at temperatures between −80° C. and 0° C. in at least one solvent, preferably an ethereal solvent, and brought to a temperature between 10° C. and 40° C. and held at this temperature for 0.5 to 4 hours, the temperature preferably being between 15° C. and 30° C. and the residence time between 0.5 and 2 hours. Thereafter, the mixture can be brought to the desired temperature for reaction with the compounds B and C.

The imidazolo-containing phosphinoborane compounds of the general formulae I and II serve as precursors for preparing optically active ligands (carbenes) of the general formulae III and IV:

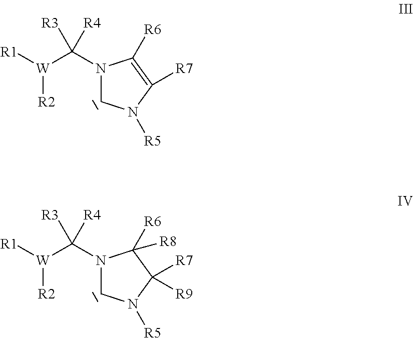

where W is phosphorus (P) or phosphinoborane (P-BH₃) and the further definitions have already been described for the general formulae I and II.

The invention further provides optically active ligands of the general formulae V and VI:

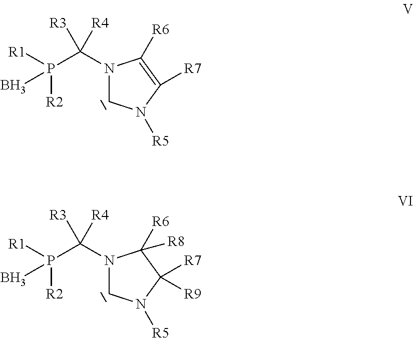

in which
R1 and R2 are different radicals and are selected from the group consisting of alkyl and alkyl (variant α)
or
R1 and R2 are different radicals and are selected from the group consisting of alkyl and aryl (variant β),
R3 and R4 are each identical or different radicals selected from the group consisting of hydrogen, alkyl and aryl,
R5 is alkyl or aryl,
R6 and R7 are each identical or different radicals selected from the group consisting of hydrogen, alkyl, aryl and a 6-membered aliphatic or aromatic ring,
R8 and R9 are each independently hydrogen or alkyl.

The preferences for the R1 to R9 radicals correspond to the preferences detailed above for the general formula I or II.

Particular preference is given to both enantiomers of the following optically active ligands:

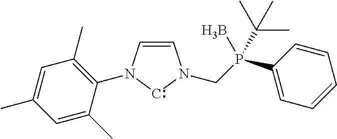

1-((tert-butyl(phenyl)phosphinoboranyl)methyl)-3-mesitylimidazol-2-ylidene

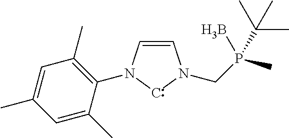

1-((tert-butyl(methyl)phosphinoboranyl)methyl)-3-mesitylimidazol-2-ylidene

The invention further relates to a process for preparing compounds of the general formula III or IV

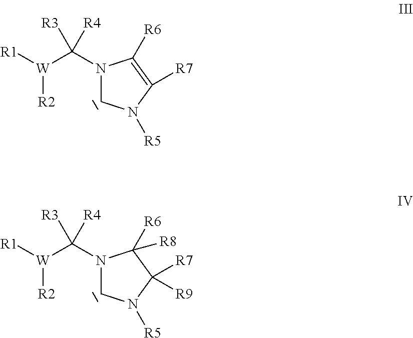

in which
W is phosphorus (P) or a phosphinoborane (P-BH₃),
R1 and R2 are different radicals and are selected from the group consisting of alkyl and alkyl (variant α)
or
R1 and R2 are different radicals and are selected from the group consisting of alkyl and aryl (variant β),
R3 and R4 are each identical or different radicals selected from the group consisting of hydrogen, alkyl and aryl,
R5 is alkyl or aryl,
R6 and R7 are each identical or different radicals selected from the group consisting of hydrogen, alkyl, aryl and a 6-membered aliphatic or aromatic ring,
R8 and R9 are each independently hydrogen or alkyl,
   which comprises converting compounds of the general formula I or II using in each case at least one strong base and an ethereal or other aprotic solvent at a temperature of −80 to +20° C. to compounds of the general formula III or IV (step (ii)), if W in the general formula III or IV is phosphinoborane, the compound of the general formula I or II, before or after step (ii), is deprotected in the presence of at least one reagent in the form (a) of a tertiary or secondary amine, or (b) of polymer-bound amine and at least one solvent at a temperature of +20° C. to +100° C. for 1 to 200 hours; if the compound of the general formula I or II is deprotected before step (i), the reagent used may also be (c) a noncoordinating acid (step (i)).

The reagent (a) used in step (i) is preferably DABCO (diazabicyclo[2.2.2]nonane), N-methylpyrrolidine, morpholine or diethylamine. The reagent (b) used is advantageously N-methylpiperidinopolystyrene, as described by Sayalero et al. in Synlett, 2006, 2585. The reagent (c) used is advantageously methanesulfonic acid or tetrafluoroboric acid.

The solvent used in step (i) is preferably a stable solvent or mixtures of such solvents, for example ethereal, halogenated or aromatic solvents such as THF, diethyl ether, tert-butyl methyl ether, dibutyl ether, toluene, hexane, chlorobenzene, chloroform, preferably THF, diethyl ether, chlorobenzene, chloroform.

The reaction temperature in step (i) is preferably 0° C. to 80° C., especially 10° C. to 50° C.

The reaction time of step (i) is typically 5 to 100 hours, preferably 10 to 80 hours. General information can be found, for example, in (a) Imamoto, T.; Kusumoto, T.; Suzuki, N.; Sato, K. J. Am. Chem. Soc. 1985, 107, 5301; (b) Imamoto, T.; Oshiki, T.; Onozawa, T.; Kusumoto, T.; Sato, K. J. Am. Chem. Soc. 1990, 112, 5244; (c) Stoop, R. M.; Mezzetti, A. Organometallics 1998, 17, 668-675; (d) Yang, H.; Lugan, N.; Mathieu, R. Organometallics 1997, 16, 2089-2095; (e) Stoop, R. M.; Bauer, C.; Setz, P.; Wörle, M.; Wong, T. Y. H.; Mezzetti, A. Organometallics 1999, 18, 5691-5700; (f) Maienza, F.; Spindler, F.; Thommen, M.; Pugin, B.; Malan, C.; Mezzetti, A. J. Org. Chem. 2002, 67, 5239-5249; (g) Brisset, H.; Gourdel, Y.; Pelton, P.; Le Corre, M. Tetrahedron Lett. 1993, 34, 4523; (h) Tsuruta, H.; Imamoto, T. Synlett 2001, 999-1002; (i) McKinstry, L.; Livinghouse, T. Tetrahedron Lett. 1994, 35, 9319. (j) McKinstry, L.; Livinghouse, T. Tetrahedron Lett. 1994, 50, 6145.

Alternatively, the process can also be performed with polymer-bound reagents. General information on this subject can be found by the person skilled in the art in: Sayalero, S; Pericàs, M. A. Synlett 2006, 2585-2588.

In step (ii), typical strong bases which are known to those skilled in the art and preferably have a pKB of at least 14 are used. For example, KOt-Bu, KOEt, KOMe, KOH, NaOt-Bu, NaOEt, NaOMe, NaOH, LiOH, LiOtBu, LiOMe, especially KOt-Bu, KOEt, KOMe, NaOt-Bu, NaOEt, NaOMe, are used.

In step (ii), all ethereal or other aprotic solvents known to those skilled in the art can be used, for example THF, diethyl ether, tert-butyl methyl ether, dibutyl ether, toluene or mixtures thereof.

The reaction time of step (ii) is typically 1 minute to 10 hours, preferably 5 minutes to 5 hours, especially 10 minutes to 1 hour.

The reaction temperature in step (ii) is preferably -60 to 40° C., especially -20 to 30° C.

The invention further relates to transition metal complexes comprising, as ligands, a compound of the general formula V or VI.

The transition metal complexes correspond to the general formula VII or VIII:

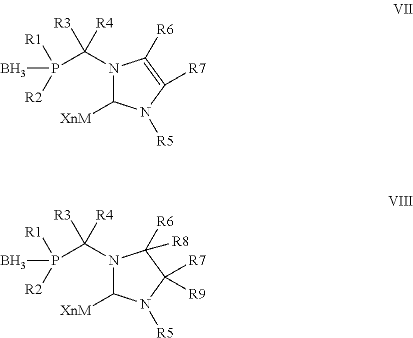

The transition metals (M) used are preferably metals of groups 8 to 11, especially Ru, Fe, Co, Rh, Ir, Ni, Pd, Pt, Ag, Cu or Au, more preferably Ru, Rh, Ir, Ni, Pd.

X represents further, optionally different, ligands, preferably cod (cyclooctadiene), norbornadiene, Cl, Br, I, CO, allyl, benzyl, Cp (cyclopentadienyl), PCy₃, PPh₃, MeCN, PhCN, dba (dibenzylideneacetone), acetate, CN, acac (acetylacetonate), methyl and H, especially cod, norbornadiene, Cl, CO, allyl, benzyl, acac, PCy₃, MeCN, methyl and H. n varies between 0 and 4 and is correspondingly dependent on the transition metal selected.

The definitions and preferences for the R1 to R9 radicals correspond to the preferences for the compounds of the general formulae III and of the general formulae I and II.

The present invention further relates to a process for preparing transition metal complexes of the general formula IX and X

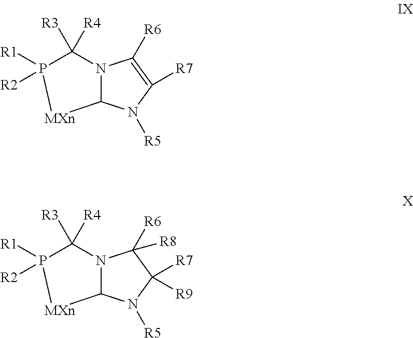

in which
R1 and R2 are different radicals and are selected from the group consisting of alkyl and alkyl (variant α)
or
R1 and R2 are different radicals and are selected from the group consisting of alkyl and aryl (variant β),
R3 and R4 are each identical or different radicals selected from the group consisting of hydrogen, alkyl and aryl,
R5 is alkyl or aryl,
R6 and R7 are each identical or different radicals selected from the group consisting of hydrogen, alkyl, aryl and a 6-membered aliphatic or aromatic ring,
R8 and R9 are each independently hydrogen or alkyl,
   which comprises
   either

(a) reacting optically active ligands of the general formula III or IV with metal complexes at a temperature of -80° C. to +120° C. using at least one solvent for 5 minutes to 72 hours;

if W in the general formula III and IV is phosphinoborane, the compound of the general formula III or IV, before or after step (a), is deprotected in the presence of at least one reagent in the form of a tertiary, secondary or polymer-bound amine at a temperature of +20° C. to +100° C. for 1 to 200 hours,

or
(b) reacting imidazolo-containing phosphinoborane compounds of the formula I or II with metal complexes using in each case at least one strong base and an ethereal or other aprotic solvent at a temperature of -80° C. to +120° C. for 5 minutes to 72 hours, and therebefore or thereafter reacting them with at least one reagent in the form of a tertiary, secondary or polymer-bound amine and at least one solvent at a temperature of +20° C. to +100° C. for 1 to 200 hours.

The selection of suitable metal complexes, especially the leaving ligands thereof, can be undertaken by the person skilled in the art by routine tests. Examples of useful metal complexes include [Ir(cod)Cl]₂, [Rh(cod)Cl]₂, [Ir(norbornadiene)Cl]₂, [Rh(norbornadiene)Cl]₂, Rh(cod)acac, [RhCl(C₈H₁₄)₂]₂, Rh(cod)(methallyl), Rh(cod)₂X, Rh(norbornadiene)₂X, Ir(cod)₂X (where X=BF₄, CIO₄, CF₃SO₃, CH₃SO₃, HSO₄, B(phenyl)₄, B[bis(3,5-trifluoromethyl)phenyl]₄, PF₆, SbCl₆, AsF₆, SbF₆), Rh(OAc)₃ Rh(acac)(CO)₂, Rh₄(CO)₁₂, RhCl(PPh₃)₃, [RhCl(CO)₂]₂, RuCp₂, RuCp(CO)₃, [RuI₂(cymene)]₂, [RuCl₂(benzene)]₂ Ru(cod)(methallyl)₂, RuCl₂(PCy₃)₂CHPh, (PCy₃)Cl₂RuCl(OiPrO-Ph), RuCl₂(C₂₁H₂₄N₂)(C₁₅H₁₀)(PCy₃), [Pd(allyl)Cl]₂, Pd₂(dba)₃, Pd(dba)₂, Pd(PPh₃)₄, Pd(OAc)₂, PdCl₂(MeCN)₂, PdCl₂(PhCN)₂, Pd(cod)CIMe, Pd(tmeda)Me₂, Pt(cod)Me₂, Pt(cod)Cl₂, SnCl₂, CuCl, CuCl₂, CuCN, Cu(CF₃SO₃)₂, [Ni(allyl)Cl]₂, Ni(cod)₂. Preference is given to [Ir(cod)Cl]₂, [Rh(cod)Cl]₂, [Ir(norbornadiene)Cl]₂, [Rh(norbornadiene)Cl]₂, Rh(cod)acac, Rh(OAc)₃, Rh(cod)₂X, Rh(norbornadiene)₂X, Ir(cod)₂X (where X=BF₄, CIO₄, CF₃SO₃, CH₃SO₃, HSO₄, B(phenyl)₄, B[bis(3,5-trifluoromethyl)phenyl]₄, PF₆, SbCl₆, AsF₆, SbF₆), Rh(acac)(CO)₂, [RhCl(CO)₂]₂, RuCp₂, RuCp(CO)₃, [RuCl₂(cymene)]₂, RuCl₂(PCy₃)₂CHPh, (PCy₃)Cl₂RuCl(OiPrO-Ph), Ru(cod)(methallyl)₂, [Pd(allyl)Cl]₂, Pd₂(dba)₃, CuCN, Cu(CF₃SO₃)₂, [Ni(allyl)Cl]₂, Ni(cod)₂ especially [Ir(cod)Cl]₂, [Rh(cod)Cl]₂, Rh(cod)acac, Rh(cod)₂X, Rh(norbornadiene)₂X, Ir(cod)₂X (where X=BF₄, CIO₄, CF₃SO₃, CH₃SO₃, HSO₄, B(phenyl)₄, B[bis(3,5-trifluoromethyl)phenyl]₄, PF₆, SbCl₆, AsF₆, SbF₆), Rh(acac)(CO)₂, [RuCl₂(cymene)]₂, RuCl₂(PCy₃)₂CHPh, Ru(cod)(methallyl)₂, [Pd(allyl)Cl]₂, Cu(CF₃SO₃)₂, [Ni(allyl)Cl]₂, Ni(cod)₂.

In option (a), the reaction temperature is advantageously −80° C. to +120° C., preferably 0° C. to +50° C. The reaction time is advantageously 5 minutes to 72 hours, preferably 1 to 24 hours. The solvents used may be all solvents familiar to those skilled in the art, for example THF, diethyl ether, hexane, pentane, CHCI₃, CH₂Cl₂, toluene, benzene, DMSO or acetonitrile, preference being given to THF, diethyl ether, CH₂Cl₂, toluene or hexane.

In option (b), the preferences in relation to the strong base and the ethereal or other aprotic solvent correspond to those for step (ii), and the preferences in relation to the reagent and the corresponding process parameters to those described for step (i). In option (b), the reaction temperature is advantageously −80° C. to +120° C., preferably 0° C. to +50° C. The reaction time is advantageously 5 minutes to 72 hours, preferably 1 to 24 hours.

The present invention further relates to catalysts comprising at least one complex with a transition metal, which comprises, as ligands, at least one compound of the general formula V or VI.

Preference is given to transition metals of groups 8 to 11, especially Ru, Fe, Co, Rh, Ir, Ni, Pd, Pt, Ag or Au, more preferably Ru, Rh, Ir, Ni or Pd.

Preference is given to the following catalysts, which are preparable either (variant 1) by reacting imidazolo-containing phosphinoborane compounds of the formula I or II with metal complexes using in each case at least one strong base and an ethereal or other aprotic solvent at a temperature of −80° C. to +120° C. for 5 minutes to 72 hours, and therebefore or thereafter reacting them with at least one reagent in the form of a tertiary, secondary or polymer-bound amine and at least one solvent at a temperature of +20° C. to +100° C. for 1 to 200 hours,

or

(variant 2) by reacting optically active ligands of the general formula III or IV with metal complexes at a temperature of −80° C. to +120° C. using at least one ethereal or other aprotic solvent for 5 minutes to 72 hours,

if W in the general formula III and IV is phosphinoborane, the compound of the general formula III or IV is deprotected beforehand in the presence of at least one reagent in the form of a tertiary, secondary or polymer-bound amine and of at least one solvent at a temperature of +20° C. to +100° C. for 1 to 200 hours
or

(variant 3) by dissolving the transition metal complexes of the formulae VII to X in at least one solvent.

Particular preference is given to variant (a). This variant represents the possibility of in situ synthesis of homogeneous catalysts.

The preferred reaction parameters of variants 1 and 2 are described above.

The invention further provides for the use of catalysts comprising at least one complex with a transition metal which comprises, as ligands, at least one compound of the general formula III or IV—as described above—for organic transformation reactions. Organic transformation reactions are understood to mean, for example, hydrogenation, hydroboration, hydroamination, hydroamidation, hydroalkoxylation, hydrovinylation, hydroformylation, hydrocarboxylation, hydrocyanation, hydrosilylation, carbonylation, cross-coupling, allylic substitution, aldol reaction, olefin metathesis, C-H activation and polymerization.

Particular preference is given to the use of catalysts comprising transition metal complexes comprising, as ligands, at least one compound whose direct precursor is selected from the group consisting of 1-((tert-butyl(methyl)phosphinoboranyl)methyl)-3-mesitylimidazol-2-ylidene, 3-tert-butyl-1-((tert-butyl(methyl)phosphinoboranyl)methyl)-imidazol-2-ylidene, 3-adamantyl-1-((tert-butyl(methyl)phosphinoboranyl)methyl)-imidazol-2-ylidene, 1-((tert-butyl(phenyl)phosphinoboranyl)methyl)-3-mesitylimidazol-2-ylidene, 3-tert-butyl-1-((tert-butyl(phenyl)phosphinoboranyl)methyl)imidazol-2-ylidene, 3-adamantyl-1-((tert-butyl(phenyl)phosphinoboranyl)methyl)imidazol-2-ylidene for asymmetric hydrogenation of unsaturated organic compounds.

The present invention can thus provide very inexpensive ligands, the efficiency of which is comparable to the prior art.

A particularly advantageous possibility is that of preparing homogeneous catalysts of different enantiomers which comprise robust carbene units.

### EXAMPLES

1) Synthesis of imidazolo-containing phosphinoborane compounds of the formula I

1.1) Synthesis of (R_{P})- and (S_{P})- 3-mesityl-1-[(tert-butyl(phenyl)phosphino)methyl]-imidazolidene

1.1.1) N-tert-butylimidazole 1 and N-mesitylimidazole 2

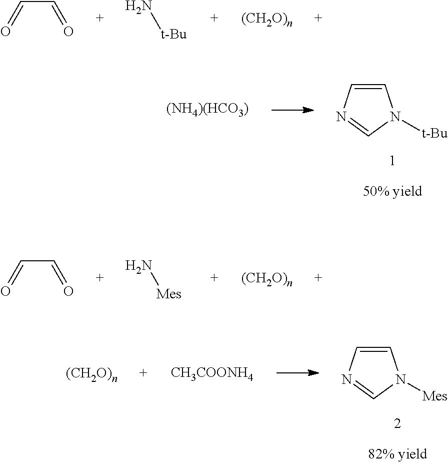

N-tert-Butylimidazole (1) and N-mesitylimidazole (2) were synthesized according to a literature method [A. J. Arduengo, III et al. U.S. Pat. No. U.S. Pat No. 6,177,575 B1].

1.1.2) rac-P(BH₃)(H)(Ph)(t-Bu)

rac-P(BH₃)(H)(Ph)(t-Bu) was prepared according to a literature method [Imamoto, T.; Oshiki, T.; Onozawa, T.; Kusumoto, T.; Sato, K. J. Am. Chem. Soc. 1990, 112, 5244].

1.1.3) 3-(halomethyl)-1-mesityl-1H-imidazol-3-ium chloride/iodide

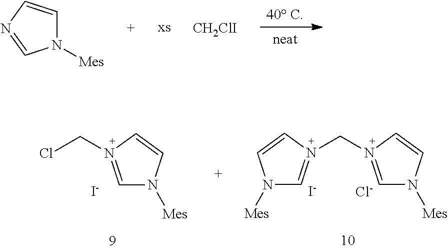

Mesitylimidazole (530 mg, 2.85 mmol) dissolved in CH₂ICl (10 ml, 137 mmol) was stirred at 40° C. for 3 days. The mixture was concentrated under reduced pressure. ¹H NMR (CDCl₃) analysis of the residue shows a mixture of 9 (85%) and 10 (15%). The white solid was washed with Et₂O (3×5 ml), suspended in CHCl₃ (5 ml), and filtered through a frit. 388 mg (38% yield) of a white powder were obtained. On the basis of NMR and elemental analysis, the product consists of an approximately 1:1 mixture of chloromethyl- and iodomethylimidazolium salts.

Anal. calcd. for C13H₁₆I₂N₂ (9-I): C, 34.39; H, 3.55; N, 6.17. Anal. calcd. for C₁₃H₁₆CIIN₂ (9-CI): C, 43.06, H, 4.45; N, 7.72. Found: C, 38.46; H, 4.00; N, 6.84. Alternatively, the crude mixture of 9 and 10 can be purified by means of column chromatography (3:1 acetone:EtO; Rf =0.31).

9-I. ¹H NMR (CDCl₃): δ 10.30 (1H, imide), 8.11 (m, 1H, imide), 7.23 (m, 1H, imide), 7.03 (2H, Ar), 6.72 (2H, CH₂), 2.36 (3H, Me), 2.14 (3H, Me), 2.12 (3H, Me) ppm. EA calcd. for C₁₃H₁₆l₂N₂ (9-l): C, 34.39; H, 3.55; N, 6.17. Found: C, 34.82; H, 3.61; N, 6.21.

1.1.4) 1-((tert-butyl(phenyl)phosphinoboranyl)methyl)-3-mesityl-1H-imidazol-3-ium iodide (8).

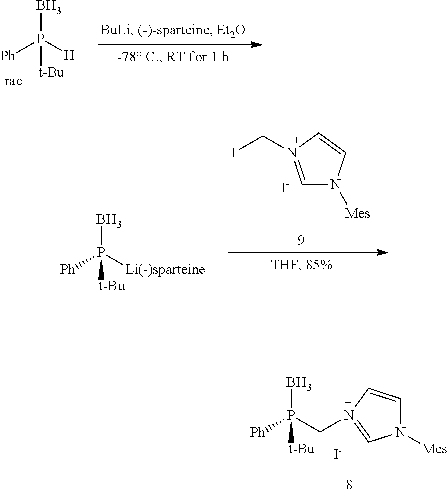

Racemic (BH₃)PH(Ph)(t-Bu) (498 mg, 3 mmol) was added to a solution of (−)-sparteine (914 mg, 3.9 mmol) in Et₂O (50 ml) under argon. Once the mixture had been cooled to −78° C., n-BuLi (2.6 M in toluene, 1.2 ml, 3 mmol) was added via a syringe. The reaction mixture was warmed to room temperature and stirred at this temperature for 1 h. Once the mixture had been cooled again to −78° C., a solution of 9-l (1.09 g, 2.4 mmol) in THF (50 ml) was added under argon. The reaction mixture was then warmed slowly to −20° C. and stirred at this temperature for 16 h. Thereafter, the mixture was washed with 5% aqueous sulfuric acid (60 ml), the aqueous phase was extracted with CH₂Cl₂ (3×50 ml), and the combined organic phases were washed with water (50 ml) and saturated sodium chloride solution (50 ml), dried over MgSO₄ and concentrated under reduced pressure, in order to obtain 850 mg (71% yield) of a white solid. ¹H NMR (CDCl₃): δ9.53 (1H, imide), 8.13-8.05 (m, 2H), 7.74 (1H), 7.47 (m, 3H), 6.96 (1H, Ar), 6.86 (1H, Ar), 6.82 (1H, Ar), 6.59 (dd, J=14, 10, 1H, CH₂), 5.25 (d, J=14, 1H, CH₂), 2.21 (3H, Me), 1.92 (3H, Me), 1.54 (3H, Me), 1.21 (d, J_{P-H}=14, 9H, t-Bu). ¹³C{¹H} NMR (CDCl₃): δ 141.4 (quat.), 138.2, 134.8 (d, J_{C-P}=10), 134.1 (d, J_{C-P}=19, quat.), 132.5 (d, J_{C-P}=3), 130.2 (quat.), 129.7 (d, J_{C-P}=14), 129.1 (d, J_{C-P}=10), 124.7, 122.2 (quat.), 121.8, 121.2 (quat.), 42.7 (d, J=31, P-CH₂), 30.5 (d, J_{C-P}=31, P-CMe₃), 25.8 (d, J_{C-P}=3, CMe₃), 21.0 (Me), 17.6 (Me), 16.7 (Me). ³¹P NMR (CDCl₃): δ 37.4 (broad) ppm. EA. Calcd. for C₂₃H₃₃BIN₂P: C, 54.57; H, 6.57; N, 5.53. Found: C, 56.72; H, 6.89; N, 5.75. HRMS (ESI) m/z calcd. for C₂₃H₃₃BN₂P (BH₃−M⁺) 379.2473, found 379.2468. HRMS (ESI) m/z calcd. for C₂₃H₃₀N₂P (M⁺) 365.2141, found 365.2140.

1.1.5) Preparation of the Borane-Protected Free Carbene 11 and of the Free Ligand 1

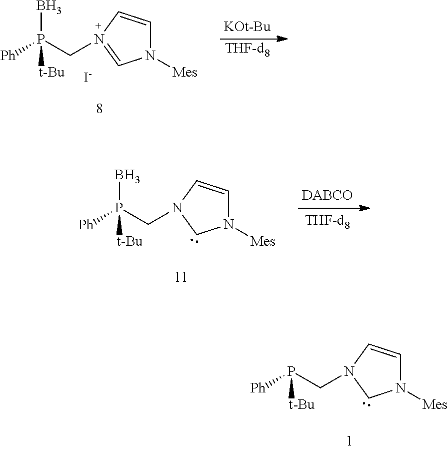

Synthesis of 11. To a solution of KOt-Bu (7 mg, 0.062 mmol) in THF-d₈ (0.5 ml) was added 8 (25.3 mg, 0.05 mmol) as a solid. The formation of a violet solution was observed immediately. The reaction was monitored by means of ¹H and ³¹P NMR (THF-d₈) spectroscopy. The reaction was complete after 3 minutes.

Selected ¹H NMR (THF-d₈) signals: δ 8.01-7.91 (m, 2H, Ar), 7.50-7.38 (m, 3H, Ar), 7.34 (d, J=2, 1H, carbene backbone), 6.89 (2H, Ar), 6.79 (d, J=2, 1H, carbene backbone), 5.23 (dd, J=14, 2, 1H, P-CH₂ carbene), 5.08 (dd, J=14, 6, 1H, P-CH₂ carbene), 2.28 (3H, Me), 1.97 (broad, 3H, Me), 1.19 (d, J=14, 9H, CMe₃). Selected ¹³C{¹H} NMR (THF-d₈) signals: δ 229.1, 137.9, 135.9, 135.5 (d, J=8), 132.3 (d, J=3), 129.4, 129.0 (d, J=10), 126.7, 125.7, 121.9, 121.6, 45.0 (d, J=34), 30.7 (d, J=29), 21.2, 18.2. ³¹P NMR (THF-d₈): 30.5 (m, P-BH₃) ppm.

Synthesis of 1. To the solution thus prepared was added DABCO (5.6 mg, 0.05 mmol) as a solid. The reaction was monitored by means of ¹H and ³¹P NMR (THF-d₈) spectroscopy. After 3 h, signals of 1 were observed in addition to the signal of the precursor 11 (ratio 1:2). After 24 h, the ratio was 2:1. After 48 h, the ratio was 7:1. About 20% by-product was likewise observed.

Selected ¹H NMR (THF-d₈) signals: δ 7.67-7.59 (m, 2H, Ar), 7.36-7.33 (m, 3H, Ar), 7.25 (d, J=2, 1H, carbene backbone), 6,93 (2H, Ar), 6.82 (d, J=2, 1H, carbene backbone), 5.04 (dd, J=14, 6, 1H, P-CH₂ carbene), 4.76 (dd, J=14, 2, 1H, P-CH₂ carbene), 2.27 (3H, Me), 1.91 (3H, Me), 1.04 (d, J=12, 9H, CMe₃). ³¹P NMR (THF-d₈): 1.4 ppm.

1.1.6) Preparation of the Imidazolium Phosphine 6-I and of the Free Ligand 1 via 6-I

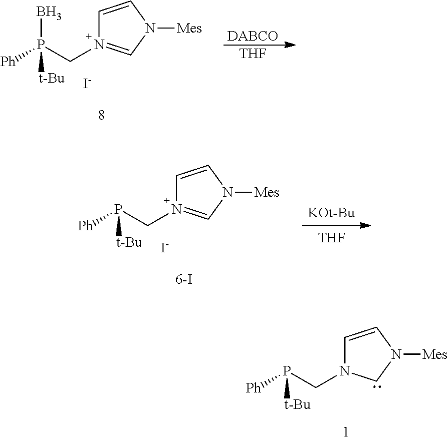

Synthesis of 6-I: To a solution of DABCO (5.6 mg, 0.05 mmol) in THF-d₈ (0.5 ml) was added 8 (25.3 mg, 0.05 mmol) as a solid, the mixture was introduced into an NMR tube and the reaction progress was monitored by means of ¹H and ³¹P NMR spectroscopy. After 24 h, the mixture comprised 88% of 6-I. After addition of further DABCO (1.7 mg, 0.015 mmol), the mixture was stirred for a further 2 days, and ³¹P NMR spectroscopy could no longer find any signals belonging to 8.

¹H NMR (THF-d₈) signals: δ 10.51 (1H, imide), 8.27 (1H, imide), 8.04-7.96 (m, 2H, Ar), 7.54 (1H, imide), 7.39-7.37 (m, 3H, Ar), 7.02-6.99 (m, 2H, Ar), 6.14 (d, J=14, 1H, P-CH₂-imide) 5.57 (dd, J=14, 12, 1H, P-CH₂-imide), 2.29 (3H, Me), 2.00 (3H, Me), 1.78 (3H, Me), 1.12 (d, J=12, 9H, CMe₃). ³¹P{¹H} NMR (THF-d₈): 11.2 ppm.

Synthesis of 1: A solution of DABCO (92.2 mg, 0.821 mmol) in THF (1 ml) was added to a suspension of 8 (320 mg, 0.632 mmol) in THF (20 ml). After three days of reaction time, no signals for the starting material could be found any longer by ³¹P NMR spectroscopy. Then KOt-Bu (78 mg, 0.695 mmol) was added as a solid, the solvent was removed under reduced pressure and the residue was extracted with pentane. After filtration through Celite, the solvent was removed under reduced pressure, and the residue was characterized by NMR spectroscopy as 1.

¹H NMR (THF-d₈): δ 7.67-7.59 (m, 2H, Ar), 7.36-7.33 (m, 3H, Ar), 7.25 (d, J=2, 1H, carbene backbone), 6.88 (2H, Ar), 6.82 (d, J=2, 1H, carbene backbone), 5.04 (dd, J=14, 6, 1H, P-CH₂-ylidene), 4.76 (dd, J=14, 4, 1H, P-CH₂-ylidene), 2.27 (3H, Me), 1.91 (3H, Me), 1.11 (3H, Me), 1.04 (d, J=12, 9H, CMe₃). ³¹P{¹H} NMR (THF-d₈): 1.4. ¹H NMR (C₆D₆): δ7.64-7.56 (m, 2H, Ar), 7.20-7.15 (m, 3H, Ar), 7.10 (d, J=2, 1H, carbene backbone), 6.79 (2H, Ar), 6.40 (d, J=2, 1H, carbene backbone), 5.01 (dd, J=14, 6, 1H, P-CH₂-ylidene), 4.72 (dd, J=14, 2, 1H, P-CH₂-ylidene), 2.51 (3H, Me), 2.16 (3H, Me), 1.31 (3H, Me), 1.04 (d, J=12, 9H, CMe₃). ¹³C{¹H} NMR (C₆D₆): δ 215.5 (s, NCN), 139.3 (quat.), 137.5 (quat.), 135.4 (d, J_{C-P}=20, Ar), 134.7 (d, J_{C-P}=19, quat.), 129.8, 129.4, 128.6 (d, J_{C-P}=8), 121.5, 119.5 (d, J_{C-P}=8), 46.5 (d, J_{C-P}=15, CH₂), 32.4 (CH₃), 30.1 (d, J_{C-P}=15, P-Cquat), 27.9 (d, J_{C-P}=14, CH₃), 21.4 (CH₃), 18.4 (CH₃). ³¹P{¹H} NMR (C₆D₆): δ 1.7 ppm.

2) Synthesis of Transition Metal Complexes of the Formula IX and X

2.1) Synthesis of [Pd(28)Cl₂] 31

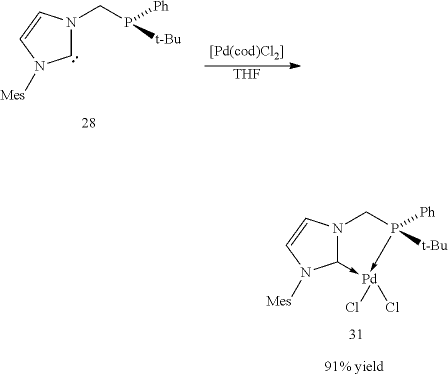

0.1 mmol of ligand 28 was dissolved under an argon atmosphere in 5 ml of THF and transferred by cannula into a suspension of 0.1 mmol of [Pd(cod)Cl₂] in 5 ml of THF. The mixture was stirred at room temperature for one day and then concentrated to dryness. Excess cod and carbene were removed by washing the resulting yellow solid with pentane, and the product was dried under reduced pressure.

Yield: 50 mg, 91%; yellow crystalline solid; ³¹P NMR (CDCl₃), δ_{P}=66.1. ¹³C NMR (CDCl₃), δ_{C} (J_{C,P}), Hz) 17.7 (s, CH₃), 18.6 (s, CH₃), 21.2 (s, CH₃), 27.0 (d, J=3.5, PC(CH₃)₃), 31.2 (d, J=10.5, PC(CH₃)₃), 45.7 (d, J=33.8, PCH₂N), 138.8-125.5 (CHₗₘ+C_{Ar}), 162.4 (s, Cₗₘ). ¹H NMR (CDCl₃), δ_{H} (J_{H,H} and J_{H,P}), Hz) 0.96 (d, J=6.6, 9H; PtBu), 1.39 (s, 3H; Me), 1.70 (s, 3H; Me), 2.02 (s, 3H; Me), 4.78 (d, J=14.8, 1H; PCH₂N), 5.41 (d, J=14.5, 1H; PCH₂N), 7.19 (s, 2H; CH_{Ar}), 7.25-7.37 (m, 5H; CHₗₘ+CH_{Ar}), 8.09 (m, 2H; CH_{Ar}). MS (ESI): m/z (%): 505 (100) [M-Cl]⁺. HRMS (TOF MS ES): Calcd. m/z 505.0786 (C₂₃H₂₉N₂PPdCl). Found m/z 505.0778.

2.2) Synthesis of [Pt(28)Cl₂] 32

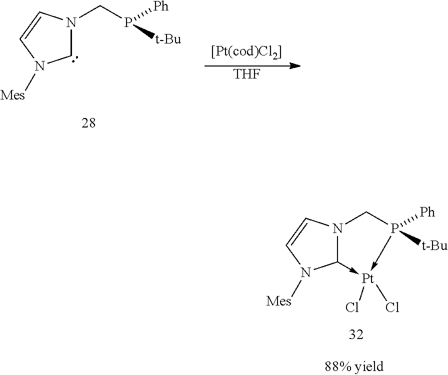

0.1 mmol of ligand 28 was dissolved under an argon atmosphere in 5 ml of THF and transferred by cannula into a suspension of 0.1 mmol of [Pt(cod)Cl₂] in 5 ml of THF. The mixture was stirred at room temperature for one day and then concentrated to dryness. Excess cod and carbene were removed by washing the resulting yellow solid with pentane, and the product was dried under reduced pressure.

Yield: 55 mg, 88%; yellow crystalline solid; ³¹P NMR (CDCl₃), δ_{P} (J_{P,Pt}), Hz) 46.1. (s+sat, J=3651). ¹³C NMR (CDCl₃), δ_{C} (J_{C,P} and J_{C,Pt}), Hz) 17.3 (s, CH₃), 18.4 (s, CH₃), 20.9 (s, CH₃), 26.6 (d, J=2.6, J=28.1, PC(CH₃)₃), 29.7 (d, J=3.5, J=18.1, PC(CH₃)₃), 45.3 (d, J=40.2, PCH₂N), 141.8-124.8 (CHₗₘ+C_{Ar}), 160.5 (s, Cₗₘ). ¹H NMR (CDCl₃), δ_{H} (J_{H,H} and J_{H,P}), Hz) 1.32 (d, J=16.9, 9H; PtBu), 1.58 (s, 3H; Me), 2.06 (s, 3H; Me), 2.22 (s, 3H; Me), 4.63 (m, 2H; PCH₂N), 7.11-8.13 (m, 9H; CHₗₘ+CH_{Ar}). MS (ESI): m/z (%): 595 (100) [M-Cl]+. HRMS (TOF MS ES): Calcd. m/z 594.1399 (C₂₃H₂₉N₂PPtCl). Found m/z 594.0505.

2.3) Synthesis of [Ni(28)(allyl)]Cl 33

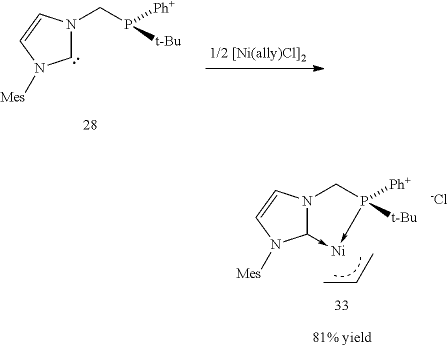

0.075 g (0.28 mmol) of [Ni(allyl)Cl]₂ and 0.212 g (0.58 mmol) of ligand 28 were weighed together into a Schlenk tube and admixed with 15 ml of hexane while stirring. After stirring at room temperature for three hours, the precipitated complex was filtered off and washed three times with 10 ml of pentane, and the product was dried under reduced pressure.

Yield: 0.227 g, 81%; yellow crystalline solid; ³¹P NMR (CDCl₃), δ_{P}=74.3 (50%), 72.4 (50%). MS (ESI): m/z (%): 463 (100) [M-Cl]+. HRMS (TOF MS ES): Calcd. m/z 463.1807 (C₂₆H₃₄N₂PNi). Found m/z 463.0682.

2.4) Synthesis of [Ni(28)allyl]B(Ar^{f})₄ 34

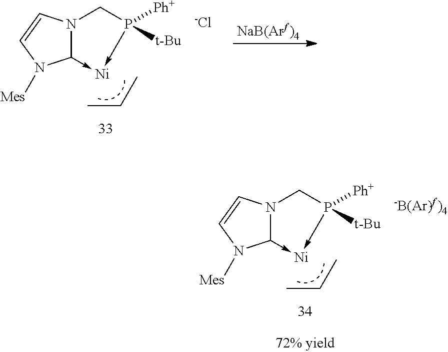

55 mg (0.11 mmol) of complex 33 and 102 mg (0.12 mmol) of NaB(Ar^{f})₄ were weighed together into a Schlenk tube and cooled to −78° C. 10 ml of diethyl ether cooled to the same temperature were transferred by cannula into this mixture with stirring, and the mixture was warmed slowly to room temperature while stirring. The solution was filtered and concentrated to dryness under reduced pressure.

Yield: 0.105 g, 72%; yellow crystalline solid; ³¹P NMR (CDCl₃), δ_{P}=78.8 (50%), 77.0 (50%). ¹³C NMR (CDCl₃), δ_{C} (J_{C,P}), Hz) 17.2 (s, CH₃), 17.4 (s, CH₃), 21.1 (s, CH₃), 26.7 (d, J=6.0, PC(CH₃)₃), 33.0 (s, PC(CH₃)₃), 46.4 (d, J=27.2, PCH2N), 56.9 (d, J=33.4, CH_{2,allyl}), 67.5 (s, CH_{2,allyl}), 114.6 (d, J=18.1, CH_{allyl}), 140.9-117.7 (CHₗₘ+C_{Ar}), 160.4 (s, C_{Ar}), 161.4 (s, C_{Ar}), 161.5 (s, Cₗₘ), 162.4 (s, C_{Ar}), 163.3 (s, C_{Ar}). ¹H NMR (CDCl₃), δ_{H} (J_{H,H} and J_{H,P}), Hz) 1.08 (d, J=15.8, 9H; PtBu), 1.53 (s, 3H; Me), 1.82 (s, 3H; Me), 1.91 (s, 3H; Me), 3.19 (m, 2H; CH_{2,allyl}), 4.22 (m, 2H; CH_{2,allyl}), 4.73 (m, 2H; PCH₂N), 6.93 (bs, 1H; CH_{allyl}), 7.48-7.67 (m, 12H; CHₗₘ+CH_{Ar}). MS (ESI): m/z (%): 463 (100) [M-BARF]^{+.}

3) Catalytic Applications

3.1) 1-Hexene Oligomerization with [Ni(28)allyl]B(Ar^{f})₄ 34

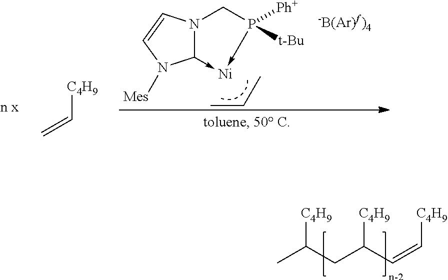

2 mg (1.5×10⁻⁶ mol) of [Ni(28)allyl]B(Ar^{f})₄ 34 were admixed with 50 ml of toluene and 5 ml of 1-hexene in a Schlenk tube. The mixture was stirred at 50° C. for one day and the oligomers were identified by GC-MS. Dimers and trimers were identified.

3.2) Ethylene Oligomerization

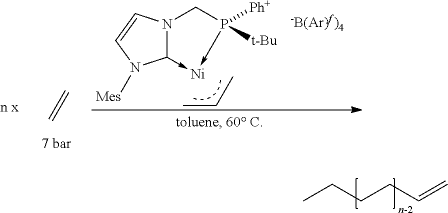

The catalyst [Ni(28)allyl]B(Ar^{f})₄ 34 (2 mg, 1.5×10⁻⁶ mol) was weighed into an autoclave in a glovebox. 50 ml of toluene were added. The autoclave was then connected to a pressure apparatus, purged repeatedly with ethylene and stirred at the desired temperature and pressure. After the desired reaction time, the oligomerization was stopped and the autoclave was opened. The resulting solution was analyzed by means of GC-MS analysis. The oligomer distribution after 12 hours was identical to the distribution after 2 hours.

Oligomers after 2 Hours (GC-MS)

| | Distribution |
|---|---|
| n | [%] |
| 2 | 16 |
| 3 | 37 |
| 4 | 34 |
| 5 | 9 |
| 6 | 3 |
| 7 | 1 |

3.3) Propylene Oligomerization

The catalyst [Ni(28)allyl]B(Ar^{f})₄ 34 (2 mg, 1.5×10⁻⁶ mol) was weighed into an autoclave in a glovebox. 50 ml of toluene were added. The autoclave was then connected to a pressure apparatus, purged repeatedly with propylene and stirred at the desired temperature and pressure. After the desired reaction time, the oligomerization was stopped and the autoclave was opened. The resulting solution was analyzed by means of GC-MS analysis. After 2 hours of reaction time, only the dimer was identified; after 24 hours of reaction time, oligomers of n=3−n=7 were identified.

3.4) Hydrogenation of Methyl α-Acetamidoacrylate

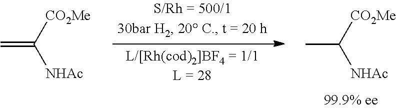

S/Rh=substrate/rhodium complex ratio [mol/mol];

t=time;

L/[Rh(cod)₂]BF₄=ratio of the NHCP ligand used (L) to the metal complex used; ee=enantiomeric excess;

ee [%]=(enantiomer 1−enantiomer 2)/(enantiomer 1+enantiomer 2); where enantiomer 1 and enantiomer 2 represent the two possible enantiomeric products.

In a glovebox, under inert conditions, 5 ml (2×10⁻⁻⁶ mol) of a stock solution of ligand 28 in CH₂Cl₂ were added to 5 ml of a solution of 2×10⁻⁶ mol [Rh(cod)₂]BF₄ in CH₂Cl₂. The mixture was stirred at room temperature for 5 min. Subsequently, 1.0 mmol of methyl α-acetamidoacrylate in 10 ml of CH₂Cl₂ was added.

The autoclave was purged three times with hydrogen, in order to remove dissolved argon. Hydrogenation was effected at 20° C. and 30 bar of H₂ for 20 h. In order to remove the catalyst, the solution was applied to a short silica gel column and eluted with CH₂Cl_{2.} The enantiomeric excess was determined by gas chromatography.

TABLE 1
Comparison with the prior art

| | | Enantiomeric |
|---|---|---|
| | Ligands | excess (ee) |
| Present invention | 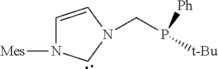 1/500 | 99.9% |
| Prior art | 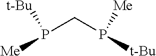 MiniPHOS 1/500 Y. Yamanoi, T. Imamoto J. Org. Chem. 1999, 64, 2988-2989 | 99.9% |
| | 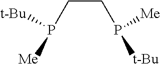 BisP* 1/500 T. Imamoto, J. Watanabe, Y. Wada, H. Masuda, H. Yamada, H. Tsuruta, S. Matsukawa, K. Yamaguchi J. Am. Chem. Soc. 1998, 120, 1635- | 98% |
| | 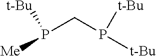 1/500 G. Hoge, H.-P. Wu, W. Kissel, D. Pflum, D. Greene, J. Bao J. Am. Chem. Soc. 2004, 126, 5966-5967 | >99% |

The prior art ligands feature complicated syntheses, in particular of the two optical antipodes. The inventive ligands can be prepared in a simple manner and with comparable efficiencies, in the form of both optical antipodes.

1. An imidazolo-containing phosphinoborane compound of the general formula I or II: 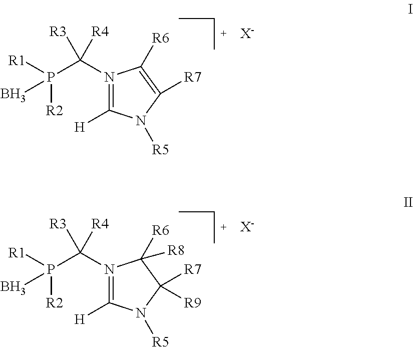
in which
R1 and R2 are different radicals and are selected from the group consisting of alkyl and alkyl (variant α)
or
R1 and R2 are different radicals and are selected from the group consisting of alkyl and aryl (variant β),
R3 and R4 are each identical or different radicals selected from the group consisting of hydrogen, alkyl and aryl,
R5 is alkyl or aryl,
R6 and R7 are each identical or different radicals selected from the group consisting of hydrogen, alkyl, aryl and a 6-membered aliphatic or aromatic ring,
R8 and R9 are each independently hydrogen or alkyl,
X is a leaving group.

2. The imidazolo-containing phosphinoborane compound of claim 1, wherein
in the case of variant α:
R1 is adamantyl, tert-butyl, sec-butyl or isopropyl, and R2 is methyl, ethyl, propyl, butyl, pentyl or hexyl,
in the case of variant β:
R1 is phenyl, tolyl, xylyl, mesityl, naphthyl, fluorenyl, anthracenyl, and R2 is adamantyl, tert-butyl, sec-butyl, isopropyl or methyl,
R3 and R4 are each independently hydrogen, methyl, ethyl or benzyl,
R5 is methyl, ethyl, isopropyl, tert-butyl, adamantyl, mesityl, phenyl, tolyl, xylyl, naphthyl, fluorenyl or anthracenyl,
R6 and R7 are each independently hydrogen or a 6-membered aromatic ring,
R8 and R9 are each independently hydrogen, alkyl or aryl,
X is a leaving group.

3. The imidazolo-containing phosphinoborane compound of claim 1, wherein
in the case of variant α:
R1 is tert-butyl and R2 is methyl or ethyl,
in the case of variant β:
R1 is phenyl and R2 is tert-butyl or methyl,
R3 and R4 are each hydrogen,
R5 is methyl, isopropyl, tert-butyl, adamantyl or mesityl,
R6 and R7 are each independently hydrogen or a 6-membered aromatic ring,
R8 and R9 are each independently hydrogen, phenyl or a (CH₂)₄ chain,
X is a leaving group.

4. A process for preparing the imidazolo-containing phosphinoborane compound of claim 1 having the general formula I or II, the process comprising reacting compounds of the general formula A 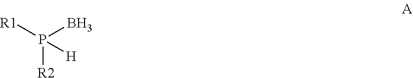
with compounds of the general formula B or C 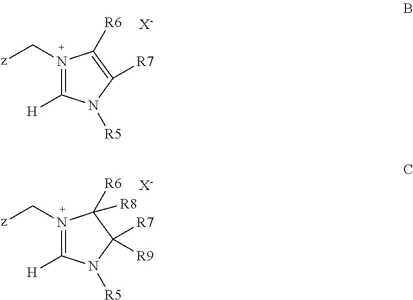
at a temperature of −80° C. to 40° C. in the presence of at least one lithium ion-containing base using one or more solvents to provide a imidazolo-containing phosphinoborane compound having the general formula I or II.

5. A process for preparing optically active imidazolo-containing phosphinoborane compounds of claim 1 having the general formula I or II, the process comprising reacting compounds of the general formula A 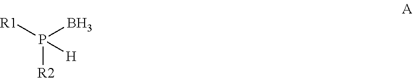
with compounds of the general formula B or C 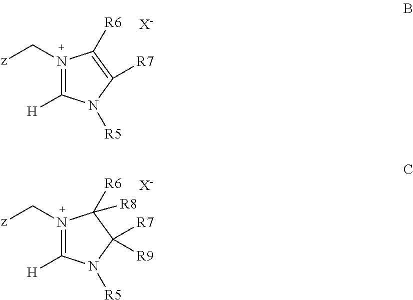
at a temperature of −80° C. to 40° C. in the presence of at least one lithium ion-containing base and of at least one chiral auxiliary using one or more solvents to provide an optically active imidazolo-containing phosphinoborane compound having the general formula I or II.

6. The process of according to claim 5, wherein the compound of the general formula A is reacted with at least one chiral auxiliary and at least one base at temperatures between −80° C. and 0° C. in at least one solvent and then brought to a temperature between 10° C. and 40° C. and held for 0.5 to 4 hours, and the resulting mixture is subsequently reacted with the compound of the general formula B or C at temperatures of −80° C. to 40° C.

7. An optically active ligand of the general formula V: 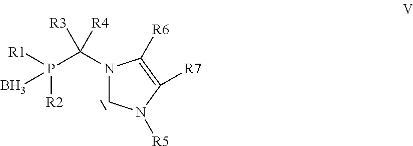
wherein
R1 and R2 are different radicals and are selected from the group consisting of alkyl and alkyl (variant α)
or
R1 and R2 are different radicals and are selected from the group consisting of alkyl and aryl (variant β),
R3 and R4 are each identical or different radicals selected from the group consisting of hydrogen, alkyl and aryl,
R5 is alkyl or aryl,
R6 and R7 are each identical or different radicals selected from the group consisting of hydrogen, alkyl, aryl and a 6-membered aliphatic or aromatic ring.

8. The optically active ligand of claim 7, wherein
in the case of variant α:
R1 is adamantyl, tert-butyl, sec-butyl or isopropyl, and R2 is methyl, ethyl, propyl, butyl, pentyl or hexyl,
in the case of variant β:
R1 is phenyl, tolyl, xylyl, mesityl, naphthyl, fluorenyl, anthracenyl, and R2 is adamantyl, tert-butyl, sec-butyl, isopropyl or methyl,
R3 and R4 are each independently hydrogen, methyl, ethyl or benzyl,
R5 is methyl, ethyl, isopropyl, tert-butyl, adamantyl, mesityl, phenyl, tolyl, xylyl, naphthyl, fluorenyl or anthracenyl,
R6 and R7 are each independently hydrogen or a 6-membered aromatic ring.

9. The optically active ligand of claim 7, wherein
in the case of variant α:
R1 is tert-butyl and R2 is methyl or ethyl,
in the case of variant β:
R1 is phenyl and R2 is tert-butyl or methyl,
R3 and R4 are each hydrogen,
R5 is methyl, isopropyl, tert-butyl, adamantyl or mesityl,
R6 and R7 are each independently hydrogen or a 6-membered aromatic ring.

10. A process for preparing compounds of the general formula III or IV, the process comprising converting compounds of the general formula I or II using at least one strong base and an ethereal or other aprotic solvent at a temperature of −80 to +20° C. to compounds of the general formula III or IV wherein if W in the general formula III or IV is phosphorus, the compound of the general formula I or II is deprotected in the presence of at least one reagent in the form (a) of a tertiary or secondary amine, or (b) of polymer-bound amine and at least one solvent at a temperature of +20° C. to +100° C. for 1 to 200 hours before or after converting the compounds of the general formula I or II; and if the compound of the general formula I or II is deprotected before converting the compounds of the general formula I or II, the reagent used may also comprise (c) a noncoordinating acid (step (i)).

11. A transition metal complex comprising a ligand selected from one or more of the general formula V or VI 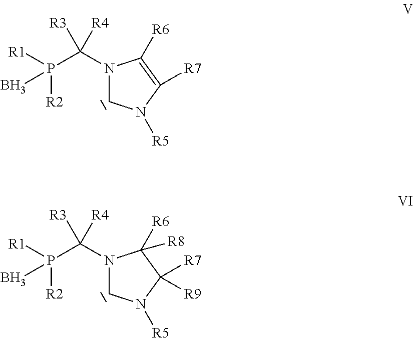
wherein
R1 and R2 are different radicals and are selected from the group consisting of alkyl and alkyl (variant α)
or
R1 and R2 are different radicals and are selected from the group consisting of alkyl and aryl (variant β),
R3 and R4 are each identical or different radicals selected from the group consisting of hydrogen, alkyl and aryl,
R5 is alkyl or aryl,
R6 and R7 are each identical or different radicals selected from the group consisting of hydrogen, alkyl, aryl and a 6-membered aliphatic or aromatic ring,
R8 and R9 are each independently hydrogen or alkyl.

12. The transition metal complex of claim 11, wherein
in the case of variant α:
R1 is adamantyl, tert-butyl, sec-butyl or isopropyl, and R2 is methyl, ethyl, propyl, butyl, pentyl or hexyl,
in the case of variant β:
R1 is phenyl, tolyl, xylyl, mesityl, naphthyl, fluorenyl, anthracenyl, and R2 is adamantyl, tert-butyl, sec-butyl, isopropyl or methyl,
R3 and R4 are each independently hydrogen, methyl, ethyl or benzyl,
R5 is methyl, ethyl, isopropyl, tert-butyl, adamantyl, mesityl, phenyl, tolyl, xylyl, naphthyl, fluorenyl or anthracenyl,
R6 and R7 are each independently hydrogen or a 6-membered aromatic ring,
R8 and R9 are each independently hydrogen, alkyl or aryl.

13. The transition metal complex of claim 11, wherein
in the case of variant α:
R1 is tert-butyl and R2 is methyl or ethyl,
in the case of variant β:
R1 is phenyl and R2 is tert-butyl or methyl,
R3 and R4 are each hydrogen,
R5 is methyl, isopropyl, tert-butyl, adamantyl or mesityl,
R6 and R7 are each independently hydrogen or a 6-membered aromatic ring,
R8 and R9 are each independently hydrogen, phenyl or a (CH₂)₄ chain.

14. The transition metal complex of claim 11, further comprising a second ligand selected from one or more of 1-(tert-butyl(methyl)phosphinomethyl)-3-mesitylimidazol-2-ylidene, 3-tert-butyl-1-(tert-butyl(methyl)phosphinomethyl)imidazol-2-ylidene, 3-adamantyl-1-(tert-butyl-(methyl)phosphinomethyl)imidazol-2-ylidene, 1-(tert-butyl(phenyl)phosphino-methyl)-3-mesitylimidazol-2-ylidene, 3-tert-butyl-1-(tert-butyl(phenyl)phosphino-methyl)imidazol-2-ylidene, and 3-adamantyl-1-(tert-butyl(phenyl)phosphinomethyl)-imidazol-2-ylidene.

15. The transition metal complex of claim 11 wherein the transition metals is selected from one or more of Ru, Fe, Co, Rh, Ir, Ni, Pd, Pt, Ag, Cu and Au.

16. A process for preparing the transition metal complex of claim 11, the method comprising
reacting optically active ligands of the general formula III or IV with metal complexes at a temperature of −80° C. to +120° C. using at least one solvent for 5 minutes to 72 hours; if W in the general formula III and IV is phosphinoborane, the compound of the general formula III or IV, before or after step (a), is deprotected in the presence of at least one reagent in the form of a tertiary, secondary or polymer-bound amine and of at least one solvent at a temperature of +20° C. to +100° C. for 1 to 200 hours,
or
reacting imidazolo-containing phosphinoborane compounds of the formula I or II with metal complexes using in each case at least one strong base and an ethereal or other aprotic solvent at a temperature of −80° C. to +120° C. for 5 minutes to 72 hours, and therebefore or thereafter reacting them with at least one reagent in the form of a tertiary, secondary or polymer-bound amine and at least one solvent at a temperature of +20° C. to +100° C. for 1 to 200 hours.

17. A catalyst comprising at least one transition metal complex comprising a ligand of the general formula V of claim 7.

18. A method of making the catalyst of according claim 17, the method comprising
(variant 1) reacting imidazolo-containing phosphinoborane compounds of the formula I or II with metal complexes using in each case at least one strong base and an ethereal or other aprotic solvent at a temperature of −80° C. to +120° C. for 5 minutes to 72 hours, and therebefore or thereafter reacting them with at least one reagent in the form of a tertiary, secondary or polymer-bound amine and at least one solvent at a temperature of +20° C. to +100° C. for 1 to 200 hours,
or
(variant 2) reacting optically active ligands of the general formula III or IV with metal complexes at a temperature of −80° C. to +120° C. using at least one solvent for 5 minutes to 72 hours,
if W in the general formula III and IV is phosphinoborane, the compound of the general formula III or IV, before or after step (a), is deprotected in the presence of at least one reagent in the form of a tertiary, secondary or polymer-bound amine and of at least one solvent at a temperature of +20° C. to +100° C. for 1 to 200 hours
or
(variant 3) dissolving the transition metal complexes of the formulae VII to X in at least one solvent.

19. A method of performing organic transformation reactions, the method comprising using a catalyst of claim 17 during an organic transformation reaction.

20. A method of performing hydrogenation, hydroboration, hydroamination, hydroamidation, hydroalkoxylation, hydrovinylation, hydroformylation, hydrocarboxylation, hydrocyanation, hydrosilylation, carbonylation, cross-coupling, allylic substitution, aldol reaction, olefin metathesis, C-H activation or polymerization, the method comprising catalyzing a hydrogenation, hydroboration, hydroamination, hydroamidation, hydroalkoxylation, hydrovinylation, hydroformylation, hydrocarboxylation, hydrocyanation, hydrosilylation, carbonylation, cross-coupling, allylic substitution, aldol reaction, olefin metathesis, C-H activation or polymerization reaction using the catalyst of claim 17.

21. A method of asymmetric hydrogenation of unsaturated organic compounds, the method comprising using a catalyst comprising the transition metal complex of claim 11 during the asymmetric hydrogenation of unsaturated organic compounds.
